(19) United States
(12) Patent Application Publication
(10) Pub. No.: US 2004/0171580 A1
(43) Pub. Date: Sep. 2, 2004

(54) Regioselectively reticulated polysaccharides

(76) Inventors: Luca Stucchi, Pavia Di Udine (IT); Alessandro Rastrelli, Padova (IT)

Correspondence Address: Mark P Levy, Thompson Hine, 2000 Courthouse Plaza NE, 10 West Second Street, Dayton, OH, 45402-1758, US

(21) Appl. No.: 10/480,208

(22) PCT Filed: Jun. 20, 2002
(86) PCT No.: PCT/EP02/06833

(30) Foreign Application Priority Data
Jun. 20, 2001 (IT) TS2001A000016

(51) Int. Cl. C08B 37/08; A61K 31/728; A61K 31/722; A61K 31/736

(52) U.S. Cl. 514/54; 514/55; 514/57; 514/60; 536/53; 536/20; 536/98; 536/111

(57) ABSTRACT

The present invention describes a class of new regioselectively reticulated polysaccharides. A process to obtain these polysaccharides, starting from naturally occurring, possibly substituted polysaccharides, is also described. The products can be used in the medical filed and in other industrial sectors.

### FIELD OF THE INVENTION

The instant invention refers to regioselectively reticulated polysaccharides. These new compounds possess particular chemical-physical characteristics and are present in diverse forms from viscous solutions to gels rendering them useful for various purposes.

### STATE OF THE ART

The literature reports diverse methods for preparing reticulated polysaccharides, which allow the attainment of materials with characteristics which are altered by varying the type of covalent bond, the functional groups involved and the polysaccharide. Depending on the type of reticulation, one can obtain polymeric networks, which, upon contact with aqueous media can result as insoluble or soluble; in the second case giving rise to systems with elevated viscosity. With this aim, polyfunctional reagents are used, as for example diepoxide, dicarbodiimide, dihydrazide, polyhydrazides, divinylsulphone, or monofunctional reagents such as for example aldehydic agents (Critical Reviews in Therapeutic Drug Carrier Systems 15(5), 513-555, 1998).

Amongst these products, a reticulated derivative of hyaluronan polysaccharide which has entered the market for clinical use is Hylan, comprising a class of derivatives which retain the biocompatibility properties of the starting polymer. The product Hylan is present in network form or as a soluble gel. In the first case the reticulation obtained with divinylsulphones exclusively involves hydroxyl groups, leaving the carboxylic and acetamidic groups of the hyaluronan intact. The realisation of the soluble gel however is obtained through the use of formaldehyde; in this case, the type of reaction precludes the possibility of further reactions for the binding of drugs.

Another reticulation strategy involving solely hydroxyl groups requires the use of diepoxides and requires very concentrated polymeric starting solutions (50-175 mg/ml) rendering the homogeneous mixture of the reagents impossible. Mono-carbodiimides, di-carbodiimides, hydrazides, dihydrazides, as reticulating agents react instead with the polysaccharide carboxylic groups. With this technique Anika have developed and marketed hyaluronan reticulates (Incert, Ossigel) for surgical use.

The technique using carbodiimide has also allowed the attainment of a heteropolysaccharide reticulate of hyaluronan and carboxymethylcellulose (Seprafilm), where both the chemical nature of the inter- and intra-chain bonds and the degree of reticulation are not however defined.

With the modification process using carbodiimides/hydrazides are also prepared polysaccharide reticulates containing a drug, where the drug is bound to the polymer through the hydrazide functional arm. The presence of this arm, can, in some cases not be ideal and interfere with the physiological processes of drug delivery in bodily areas, or release non biocompatible or bioabsorbable chemical entities in the organism.

Other reticulating agents such as dimethylolurea, ethylenoxide, polyaziridine, polyisocyanates, can be used to produce polysaccharide reticulates; the products obtained with these agents have however chemical structures which are not defined.

According to another approach, the polysaccharide is partially oxidised and the aldehyde groups formed are made to react with amino groups, for example of a second polysaccharide, in this way forming a network structure. In this case the native polysaccharide structure is however lost following the opening of the glycosidic ring (EP1011690).

Another strategy in the preparation of reticulated polysaccharides is that described in U.S. Pat. No. 5676964. The reaction involves both the carboxylic and the hydroxylic group of the hyaluronan and does not require the use of cross-linking agents but requires the presence of condensing agents. The reaction takes place randomly on both the hydroxyl groups of the N-acetyl-D-glucosamine residue and of the D-glucuronic acid residue; this renders the chemical structure of the final reticulate unpredictably variable.

In all the reticulation reactions described, the use of cross-linking or condensing agents of diverse chemical natures are required. Considering the physical nature and the high/very high viscosity of the final polysaccharide product, these agents can remain entrapped inside the network in their original, non reacted forms, or in one or more forms modified as a result of the reaction process; these potential residues withheld in the matrix can therefore interfere with the final use of the product or confer upon it undesired properties.

In the light of that reported in the literature, the need to prepare polymeric networks, well characterised from a chemical viewpoint, and also free from chemical groups foreign to the native polysaccharide, or foreign to the polysaccharide, previously modified for functional purposes, is clear. In the case of specific applications in the field of medicine, the reticulated materials appear more interesting when they have a chemical structure which allows the formation of a direct bond with biologically active molecules.

### DESCRIPTION OF THE INVENTION

Subject matter of the present invention is a class of regioselectively reticulated polysaccharides consisting of two polysaccharides, where the hydroxyl groups of the carbon atom in position 6 (carbon C-6) of the monosaccharide units of the first polysaccharide are regiospecifically esterified with the carboxylic groups of the second polysaccharide and/or with possible carboxylic groups of the first polysaccharide.

The esterification involves all or part of the carboxylic groups present in the second polysaccharide; in addition, when the first polysaccharide contains carboxylic groups, also these can be totally or partially esterified.

The carboxylic groups of the regioselectively reticulated polysaccharides not involved in the esteric bonds are in acid or salt forms; when they are in the salt form, the groups may be salified with alkali metal, alkaline earth metal, and nitrogen-containing cations. Amongst the nitrogen-containing cations are comprised those containing organic nitrogen, for example tetra- alkylammonium salts, where alkyl has 1-6 carbon atoms. Other examples are lutidinium, collidinium, imidazolium.

The first polysaccharide must contain hydroxyl groups on the carbon in position 6 (carbon C-6) of the monosaccharide units, which allow regioselective esterification, and can contain also carboxylic groups. For the purpose of the invention, the first polysaccharide is preferably selected from these which contain also carboxylic groups. When the polysaccharide contains carboxylic groups, these are in the form of acids or salts, in the latter case being preferably salified with alkaline metal or alkaline earth metal cations, with organic cations or nitrogen-containing inorganic cations. Polysaccharides (first polysaccharide) which contain hydroxyl groups on carbon C-6 of the monosaccharide units and contain also carboxylic groups are glycosaminoglycans, xanthan, cellulose carboxylate derivatives of chitin, of amylose, of dextran with degrees of substitution less than 100%, vegetable gums. Specific examples are hyaluronan, chondroitin 4-sulphate, dermatan sulphate, heparin, carboxymethylcellulose, carboxymethylchitin, carboxymethylamylose, carboxymethylguar, carboxymethyldextran; gum arabic, gum tragacanth, ghatti gum. Polysaccharides (first polysaccharide) which contain hydroxyl groups on carbon C-6 of the monosaccharide unit and do not also contain carboxylic groups are β-glucans, pullulan, curdlan, gellan, succinoglycan, galactomannans, glucomannans.

For the purpose of the present invention, the first polysaccharide is preferably selected from the group which consists of hyaluronan, xanthan, carboxymethylcellulose with degrees of substitution less than 100%, preferably comprised of between 0-50%, carboxymethylchitin with degrees of substitution less than 100%, preferably comprised of between 0-50%, carboxymethylamylose with degrees of substitution less than 100%, preferably comprised of between 0-50%, carboxymethylguar with degrees of substitution less than 100%, scleroglucan, laminaran.

The second polysaccharide is a carboxylate polysaccharide: this must contain carboxylic groups on the monosaccharide units that constitute it. Said polysaccharide is present in acid or salt form; in the latter the carboxylic groups are salified with alkaline metal, alkaline earth metal cations or nitrogen-containing cations. Polysaccharides which contain carboxylic groups on the monosaccharide units (second polysaccharide) are: glycosaminoglycans, xanthan, carboxylate derivatives of cellulose, of chitin, of amylose, of dextran, alginates, vegetable gums, pectins with degrees of esterification less than 100%. Examples of polysaccharides which fall into this group are: hyaluronan, chondroitin 4-sulphate, chondroitin 6-sulphate, dermatan sulphate, heparin; carboxymethylcellulose, carboxymethylchitin, carboxymethylamylose, carboxymethylguar, carboxymethyldextran; gum arabic, gum tragacanth, ghatti gum. For the purpose of the present invention the second polysaccharide is preferably selected from the group consisting of hyaluronan, xanthan, alginate, carboxymethylcellulose, carboxymethylchitin, carboxymethylamylose, carboxymethylguar.

The first and second polysaccharides have weight average molecular weight (MW, determination by HPSEC and/or coupled with a molecular size detector for example light diffusion) preferably comprised between 500 and 3000000 or more preferably between 800 and 1500000. The weight average molecular weights of the two polysaccharides can be different or the same. In the case of different weights, usually the weight average molecular weight of the first polysaccharide is lower than that of the second polysaccharide.

The first and second polysaccharides which make up the reticulated polysaccharide according to the present invention can be identical. In this case the preferred polysaccharide is hyaluronan. The first and second polysaccharides, both hyaluronan, have the same or different weight average molecular weights. When the two polysaccharides are different, then the first is preferably a β-glucan, chosen from between either laminaran or scleroglucan, the second is preferably hyaluronan.

Both the first and second polysaccharides which make up the reticulated derivative can further be substituted on the free positions, or on the hydroxyl groups, the carboxylic groups, the amino groups, where present. The substitution can be performed through the introduction of structurally different chemical groups which confer important functional properties for the use of the reticulated products of the invention. For example, these polysaccharides can be substituted with drugs or biologically active substances, as for example medicines such as antitumoral, anti-inflammatory or wound healing substances.

Regarding the regioselective esterification of the hydroxyl groups, this can involve all the hydroxyl groups (100% esterification) or just a part of these. These regioselectively reticulated polysaccharides where the number of esterified hydroxyl groups is comprised between 0.01% and 70% are preferred.

Another subject of the present invention is the process for the preparation of reticulated polysaccharide. The procedure is characterised by the following steps: a) regioselective modification of the first polysaccharide through activation of the carbon C-6 of the monosaccharide units of the polysaccharide; b) formation of ester bonds between the carboxylic group of the second polysaccharide and the C-6 atom of the first polysaccharide, regioselectively activated, obtained in a).

In step a), the activation of the carbon C-6 is attained by replacing the hydroxyl group in C-6 with a suitable leaving group. Preferably the activation is obtained by regioselectively halogenating the carbon C-6 of the monosaccharidic units of the polysaccharide. Said halogenation can be performed as follows: the first polysaccharide is suspended in an appropriate organic solvent and kept agitated for 1-5 hours at 25-100° C.; then the hydroxyl groups of carbon C-6 are made to react with a halogenating agent in organic solvent at a temperature of between −20° C. and 70° C. with agitation for 1-18 hours. The pH of the reaction mixture can be adjusted to values of between 9-11. At the end of the regioselective halogenation reaction the mixture is neutralised and the product recovered and purified according to known methods. The halogenating agent is selected from the group consisting of: methanesulphonyl bromide, methanesulphonyl chloride, p-toluenesulphonyl bromide, p-toluenesulphonyl chloride, thionylcloride, thionylbromide; alternatively, one could use, for example, oxalyl bromide, oxalyl chloride, phosgene, bis(trichloromethyl) carbonate also in appropriate mixtures according to the art. The solvents which can be used are the aprotic solvents such as dialkylsulphoxide, dialkylcarboxamides, in particular C1-C6-dialkylsulphoxides, as for example dimethylsulphoxide, and C1-C6 dialkylamides of C1-C6 aliphatic acids, as for example N,N-dimethylformamide, N,N-diethylformamide, N,N-dimethylacetamide, N,N-diethylacetamide. The preferred solvents are N,N-dimethylformamide, dimethylsulphoxide, N-methylpyrolidone.

When the first polysaccharide to be activated contains carboxylic groups these are in acid or salt form, preferably in salt form, still more preferred are salts with organic nitrogen-containing cations, for example tetralkylammonium salts.

Further details for the regioselective halogenation of polysaccharides containing also a carboxylic group are described in WO99/18133.

In step a), the C-6 carbon atoms can also by activated by reactions other than halogenation: in fact any reactions selectively allowing the introduction of a good leaving group on the C-6 carbon atom of the monosaccharide unit can in principle be applied for this purpose: as an example, C-6 O-alkylsulphonates or C-6 O-arylsulphonates of polysaccharides can be produced by treating the polysaccharide in organic solvent with the required amount of alkyl- or arylsulphonyl halide in the presence of a base catalyst at low temperature, e.g. below room temperature.

In step b) the first regioselectively activated polysaccharide obtained in a), is suspended, preferably in the acid form, in an organic solvent or in a mixture of organic solvents and then mixed with the second polysaccharide suspended in the same solvent, in the presence of a basic agent. The reaction is carried out at a temperature of between 25 and 90° C. for 1-100 hours. The product is recovered according to classical techniques, such as for example, precipitation, filtration, desiccation, lyophilization. The reaction may also require the addition of catalysts. The ideal solvents are the aprotic solvents such as dialkylsulphoxide, dialkyl carboxyamides, in particular C1-C6-dialkylsulphoxides, as for example dimethylsulphoxide, and the C1-C6 dialkylamides of C1-C6 aliphatic acids, such as for example N,N-dimethylformamide, N,N-diethylformamide, N,N-dimethylacetamide, N,N-diethylacetamide. The solvent is preferably selected from N,N-dimethylformamide, dimethylsulphoxide, N-methylpyrrolidone. The basic agent is chosen from either inorganic or organic bases. For example, particularly suitable are the alkaline metal carbonates, pyridine and its substituted forms, such as, for example, dimethylaminopyridine, morpholine, oxazoline, triazoles, tetrazoles, quinoline and also substituted for example, with amine and methyl groups. Base precursors can also be used.

Other reactions, different from these described which allow however the development of steps a) and b) can be applied.

In the reticulated products obtained by applying steps a) and b) to the preparation process, the free carboxylic groups, not involved in ester bonds can possibly be salified according to one of the known procedures.

Regarding the starting polysaccharides, the first (when containing carboxylic groups) and the second preferably have the carboxylic groups salified with organic nitrogen-containing cations; and, still more preferably, with tetra alkylammonium salts, wherein the alkyl groups contain from 1 to 6 carbon atoms. In most cases tetrabutylammonium carboxyl polysaccharide is used. It is possible to prepare these salts by reacting a sodium salt of the polysaccharide or its free acid form in aqueous solution with a sulphonic resin salified with an appropriate quaternary ammonium base.

Variations in the reaction conditions, such as the concentration of the starting polysaccharide solution, the ratio between the first and second polysaccharide, the ratio between the reagents and the single polysaccharides, the reaction temperature, the duration of the reaction, allow the modulation of the degree of reticulation.

The process of preparing reticulated polysaccharides presents the essential characteristics to allow a regioselective reaction. In fact, the reaction proceeds solely on hydroxyl groups present on C-6 of the monosaccharide residue of the first polysaccharide, which are appropriately activated so as to be the only hydroxyl groups of the monosaccharide units capable of reacting with the carboxylic group to form an ester bond. With this process a regioselectively reticulated polysaccharide product which is chemically well defined is obtained. A further advantage of the process in the present invention lies in the fact that said procedure allows the production of regioselectively reticulated polysaccharides in the absence of other agents, such as for example cross-linking agents and/or condensating agents commonly used in state of the art processes for the preparation of reticulated polysaccharides. This furnishes pure regioselectively reticulated polysaccharides, i.e. free from contaminants originating from agents used in the reaction process. These contaminants could interfere with the final use of the reticulate; in particular in the case of use in the field of medicine these could constitute toxic or noxious components or convey side effects, for example inflammatory activity.

By varying the type of polysaccharide and the degree of esterification of the hydroxyl groups on carbon C6, the reticulated polysaccharide of the invention assumes the properties of a high molecular weight polysaccharide, where for high molecular weight is intended a molecular weight value greater than the weight average molecular weights (MW) of the native polysaccharides. The regioselectively reticulated polymer of the invention when placed in contact with water or aqueous solutions swells evidently, and with mechanical agitation, gives very viscous, transparent solutions, in some cases lightly opalescent with notably complex rheologic properties. In other cases the addition of water or aqueous solutions to the reticulated polysaccharide produces aqueous gels with good mechanical characteristics. The reticulated polysaccharide is therefore presented in all possible forms from very viscous solutions to very high rigidity gels. When the reticulated polysaccharide contains substitutions with functional groups, these can permit the preparation of high viscosity systems in non aqueous solvents or in mixed solvents. These characteristics are the basis of the industrial use of the regioselectively reticulated polysaccharides of the invention.

The regioselectively reticulated polysaccharides claimed in the present invention can be used in pharmaceutical, healthcare, surgical and cosmetic fields.

Products which present the characteristics of being biocompatible and bioabsorbable can be advantageously used as biomaterials. Therefore a further subject of the invention is healthcare articles or surgical accessories comprising said products. These can therefore be used in viscosupplementation, that is in all adjuvant surgical practices, and therefore in the ophthalmic, orthopaedic, neurological sectors. Furthermore, they can find uses in the field of surgery, to block the phenomenon of the formation of adhesions, commonly following some surgical interventions, for example thoracic, abdominal, pelvic, orthopaedic, etc. These compounds can also be interesting for tissue repair, for controlled release systems or as carriers for active substances. Therefore, in these cases, they can find uses in these pathologies which require continued pharmacological treatment and in addition also for long periods with drugs which, as a result of undesired side effects or for problems with pharmacokinetics cannot be administered by the usual methods. Therefore, the subject of the invention also addresses medicaments comprising reticulated polysaccharides.

Cosmetic products comprising the reticulated polysaccharide, useful above all for topical applications, such as a hydratant, are still further subjects of the invention. The reticulated products of this invention can also be used in other industrial sectors such as in the preparation of plastic materials, composite materials, packing materials, high technology materials, adhesives, paints, industrial additives, compatibilising agents, rheologic modifiers.

Other uses for the compounds in the invention are identified in the preparation of chromatographic stationary phases, among these also chiral stationary phases. In the preparation of chromatographic stationary phases, these reticulates are used as such, after having been ground or shaped into beads and after having eventually been selected on the basis of particle size. Therefore, constituting a further subject of the present invention are the stationary chromatographic phases comprising the regioselectively reticulated polysaccharides of the invention. These stationary phases are prepared according to the processes known by engineers in the field and can be used in thin layer chromatography, in liquid chromatography, for example HPLC, batch chromatography, “simulating moving bed” (SBM) mediated chromatography, supercritical fluid chromatography (SFC).

The following examples are non-limiting illustrations of the invention.

EXPERIMENTAL PART

### EXAMPLE 1

Method of Determination of the Weight Average Molecular Weight (MW) of Hyaluronan

The mean molecular weight is determined by HP-SEC (High Performance Size Exclusion Chromatography). The analysis conditions are: Chromatographic system: HPLC Jasco PU-880 with Rheodyne 9125 injector. Column: TSK PWxl G6000+G5000+G3000 (TosoHaas) 300 mm×7.8 mm ID, 13, 10, 6 μM particle size; Temperature 40° C. Mobile phase: 0.15 M NaCl. Flow rate: 0.8 ml/min. Detector: LALLS CMX-100 (TSP Chromatix), Po=300-400 mV; Differential refractive index 410 (Waters), Sensitivity 128×; Temperature 32° C. Volume injected: 100 μl. The products are solubilised in 0.15 M NaCl at a concentration of approx. 1.0 mg/ml and are agitated for 12 hours. The solutions are filtered through a 0.45 μm porosity filter (Millipore) and then injected into the chromatographic system. The analysis allows the determination of the MW (weight average molecular weight), Mn (number average molecular weight), Pl (polydispersity). The concentrations of the polysaccharide solution are verified using the integral of the refractive index. The tetrabutylammonium hyaluronan (samples used in examples 3-6, 8-14) is analysed after ion exchange between tetrabutylammonium and sodium, the MW determination is then performed on the corresponding sodium salt.

### EXAMPLE2

Method of Determination of the Weight Average Molecular Weight (MW) of Laminaran

The weight average molecular weight is determined by HP-SEC (High Performance Size Exclusion Chromatography). The analysis conditions are: Chromatographic system: HPLC Jasco PU-880 with Rheodyne 9125 injector. Column: TSK PW×l G3000+G2500 (TosoHaas) 300 mm×7.8 mm ID, 13, 10, 6 μm particle size; Temperature 40° C. Mobile phase: 0.15 M NaCl. Flow rate: 0.5 ml/min. Detector: Differential refractive index 410 (Waters), Sensitivity 128×; Temperature 32° C. Volume injected: 100 μl. The products are solubilised in 0.1 M NaCl at a concentration of approx. 1.0 mg/ml and are agitated for 12 hours. The solutions are filtered through a 0.45 μm porosity filter (Millipore) and then injected into the chromatographic system. The analysis allows, through prior calibration with a pullulan standard (PolymerLab UK), the determination of the MW (weight average molecular weight), Mn (number average molecular weight), Pl (polydispersity). The concentrations of the polysaccharide solutions are verified using the integral of the refractive index.

### EXAMPLE 3

Preparation of C6-bromo hyaluronan

250 mg of tetrabutylammonium hyaluronan (the sodium salt of which has MW:100000) are dissolved in 10 ml of N,N-dimethylformamide in a three-necked, refrigerated reaction flask with a refluxer, at a temperature of 80° C., under a nitrogen current, with a magnetic stirrer. The temperature was adjusted to room temperature and a further 5 ml of N,N-dimethylformamide were added. The temperature of the mixture was adjusted to 0° C. and 312 μl of thionyl bromide added. The system is left with agitation for 30 minutes and then the temperature is raised to 80° C. and left to react for a further 16 hours. The solution is cooled to room temperature, 10 ml of Milli-Q water added and neutralised with sodium hydroxide. The solvent is removed under reduced pressure and the solid obtained is washed with acetone and recovered by filtration. The product is then dissolved in 10 ml of Milli-Q water and dialysed against distilled water. The product is then recovered by lyophilization. 30 mg of product are obtained.

The product has been characterised using proton and carbon nuclear magnetic resonance spectroscopy (¹H NMR, ¹³C NMR) using deuterated water as a solvent at a temperature of 40° C. In the ¹³C spectrum, by comparison of the areas of the signals for C6 brominated (33 ppm) and that for C6 non brominated (61 ppm) it has been established that the degree of bromination is 90%.

### EXAMPLE 4

Preparation of C6-bromo hyaluronan

2 g of tetrabutylammonium hyaluronan (for which the sodium salt has MW:100000) are dissolved in 100 ml of N,N-dimethylformamide in a three-necked, refrigerated reaction flask with a refluxer, at a temperature of 60° C., under a nitrogen current and with mechanical agitation. Upon complete solubilisation, the solution is first cooled to room temperature and then to 0° C. with an ice bath. To the solution is added 101 μl of methanesulphonic acid and, in 2 aliquots, 1.5 ml of oxalyl bromide and the system left with agitation for 30 minutes. The temperature is then raised to 60° C. and left to react for 16 hours. A heterogeneous system is obtained to which 10 ml of Milli-Q water are added, then neutralized with sodium hydroxide. The system is concentrated under reduced pressure to approx. ⅕ of its volume and precipitated in methanol. The product is filtered and dispersed in water. The system is basified with sodium hydroxide and then agitated magnetically until the complete solubilisation of the product. The solution is then neutralised with hydrochloric acid and dialysed against distilled water. The product is recovered through lyophilization. 610 mg of product are obtained. The product has been characterised by proton and carbon nuclear magnetic resonance spectroscopy (¹H NMR, ¹³C NMR) using deuterated water as a solvent at a temperature of 40° C. In the ¹³C spectrum, by comparison of the area of the signal for C6 brominated (33 ppm) and that for C6 non brominated (61 ppm) it has been established that the degree of bromination is 50%.

### EXAMPLE 5

Preparation of C6-bromo hyaluronan

1 g of tetrabutylammonium hyaluronan (the sodium salt thereof has MW:100000) are dissolved in 100 ml of N,N-dimethylformamide in a three-necked, refrigerated reaction flask with a refluxer, at a temperature of 60° C., under a nitrogen current and with mechanical agitation. Upon complete solubilisation, the solution is cooled to room temperature and then to 0° C. in an ice bath. To the solution are added 50 μl of methanesulphonic acid and 754 μl of oxalyl bromide and the system left with agitation for 30 minutes. The temperature is then raised to 80° C. and the system left to react for 16 hours. A heterogeneous system is obtained to which 10 ml of Milli-Q water are added, then neutralized with sodium hydroxide. The system is concentrated under reduced pressure to approx. ⅕ of its volume and precipitated in acetone. The product is filtered and dispersed in 100 ml of Milli-Q water. The system is basified with sodium hydroxide and then agitated magnetically until the complete solubilisation of the product. The solution is then neutralised with hydrochloric acid and dialysed against distilled water. The product is recovered by lyophilization. 440 mg of product are obtained. The product has been characterised by proton and carbon nuclear magnetic resonance spectroscopy (¹H NMR, ¹³C NMR) using deuterated water as a solvent at a temperature of 40° C. In the ¹³C spectrum, by comparison of the area of the signal for C6 brominated (33 ppm) and that for C6 non brominated (61 ppm) it has been established that the degree of bromination is 70%.

### EXAMPLE 6

Preparation of C6-bromo hyaluronan

4 g of tetrabutylammonium hyaluronan (the sodium salt of which has MW:100000) are dissolved in 200 ml of N,N-dimethylformamide in a three-necked, refrigerated reaction flask with a refluxer, at a temperature of 60° C, under a current of nitrogen and with mechanical agitation. Upon complete solubilisation, the solution is cooled to room temperature and then to 0° C. in an ice bath. To the solution are added 202 μl methanesulphonic acid and, in 3 aliquots, 3.0 ml of oxalyl bromide and the system left with agitation for 30 minutes. The temperature is raised to 60° C. and left to react for 16 hours. 20 ml of Milli-Q water are added to the system, and then neutralised with 40% tetrabutylammonium hydroxide. The system is concentrated under reduced pressure to approx. ⅕ of its volume and precipitated in acetone. The product is filtered and dispersed in 150 ml of Milli-Q water. The system is basified with 40% tetrabutylammonium hydroxide and then left with magnetic stirring until the complete solubilisation of the product. The solution is then neutralised with hydrochloric acid and dialysed against distilled water. The product is recovered by lyophilization. 2.31 g of product are obtained. The product has been characterised by proton and carbon nuclear magnetic resonance spectroscopy (¹H NMR, ¹³C NMR) using deuterated water as solvent at a temperature of 40° C. In the ¹³C spectrum, by comparison of the area of the signal for C6 brominated (33 ppm) and that for C6 non brominated (61 ppm) it has been established that the degree of bromination is 50%.

### EXAMPLE 7

Preparation of C6-bromo laminaran

0.5 g of laminaran (“hot extraction” of Laminaria sp; MW: 10000, according to example 2) were dispersed in 5 ml of N,N-dimethylformamide, the system is cooled to −40° C. in an ethanol bath. To the solution are added 4 ml of methanesulphonyl bromidic acid through a dropping funnel over a period of time of over 1 hour. The temperature is then adjusted to room temperature and left to react for 2 and a half hours. The temperature is adjusted to 70° C. and left to react for 16 hours. The system is neutralised with a carbonate solution and dialysed against distilled water. The precipitate obtained is recovered and solubilised in a minimal volume of N,N-dimethylformamide and then dialysed against distilled water. The precipitate is recovered and then dried. The product has been characterised by carbon nuclear magnetic resonance spectroscopy (¹³C NMR). In the ¹³C spectrum, the signal for C6 brominated (33.77 ppm) is observed, whilst that for C6 non brominated (60.8 ppm) is absent, it has therefore been established that the degree of bromination is 100%.

### EXAMPLE 8

Preparation of Reticulated Polysaccharide

50 mg of hyaluronic acid (MW: 100000) are dissolved in 10 ml of dimethylsulphoxide in a 50 ml, three-necked reaction flask under a nitrogen current and with magnetic stirring at room temperature. In another 50 ml, three-necked reaction flask under a nitrogen current and with magnetic stirring at room temperature, are dissolved 100 mg of 6-bromo hyaluronan in acid form, prepared as in example 3 in 10 ml of dimethylsulphoxide. The two solutions are combined and left to react at room temperature and under a current of nitrogen for 45 hours in the presence of a basic agent. The solution is precipitated with methanol and the product recovered by filtration under reduced pressure. Adding to the solid 100 ml of Milli-Q water gives a heterogeneous system at acid pH. This is therefore neutralized with sodium bicarbonate thus giving a homogeneous system which is lyophilized. The product obtained swells in aqueous solutions at high concentrations.

### EXAMPLE9

Preparation of Reticulated Polysaccharide

In a 20 ml, round-bottomed flask with magnetic stirring at a temperature of 80° C., are dissolved 50 mg of C6-bromo hyaluronan, prepared as in example 6, in 10 ml of N,N-dimethylformamide. Upon complete solubilisation, the system is cooled to room temperature. In another 50 ml, three-necked flask with magnetic stirring and under a nitrogen current at a temperature of 30° C., 50 mg of tetrabutylammonium hyaluronan (the sodium salt of which has MW:100000) are dissolved in 3 ml of N,N-dimethylformamide. To the solution are then added the C6-bromo hyaluronan dissolved in N,N-dimethylformamide, 25 μl of triethylamine and a catalytic quantity of tetrabutylammonium iodide and then left to react at 30° C. with magnetic stirring and under a nitrogen current for 22 hours. The system is concentrated under reduced pressure to ⅓ of its volume and precipitated in acetone. The product is recovered by filtration and then dispersed in 50 ml of Milli-Q water. A heterogeneous system is obtained at basic pH which is neutralized with hydrochloric acid and dialysed against distilled water. The product is recovered by lyophilization. The product obtained swells in aqueous solution at high concentrations with the attainment of a very viscous system.

### EXAMPLE 10

Preparation of Reticulated Polysaccharide

50 mg of tetrabutylammonium hyaluronan (the sodium salt of which has MW:100000) are dissolved in 2 ml of dimethylsulphoxide in a 20 ml, round-bottomed reaction flask with magnetic stirring at room temperature. Upon complete solubilisation, are added 30 mg of dimethylaminopyridine and a catalytic quantity of tetrabutylammonium iodide. In another 10 ml, round-bottomed flask with magnetic stirring at a temperature of 50° C., are dissolved 50 mg of C6-bromo hyaluronan, prepared as in example 6, in 2 ml of dimethylsulphoxide. The system is cooled to room temperature and added to the tetrabutylammonium hyaluronan solution. This is left to react at room temperature with gentle agitation for 2 hours. To the system obtained are added 20 ml of Milli-Q water, neutralized with hydrochloric acid and dialysed against distilled water. The product is recovered by lyophilization. The product swells when dispersed in aqueous solutions.

### EXAMPLE 11

Preparation of Reticulated Polysaccharide

90 mg of tetrabutylammonium hyaluronan (the sodium salt of which has MW:100000) are dissolved in 5 ml of dimethylsulphoxide in a 50 ml, round-bottomed flask with magnetic stirring at room temperature. Upon complete solubilisation, 39 mg of dimethylaminopyridine and a catalytic quantity of tetrabutylammonium iodide are added. In another 10 ml, round-bottomed flask with magnetic stirring at room temperature, are dissolved 10 mg of C6-bromo hyaluronan, prepared as in example 4, in acid form, in 2 ml of dimethylsulphoxide. To the solution are added 4 mg of dimethylaminopyridine and then all is added to the tetrabutylammonium hyaluronan. This is left to react at room temperature with gentle magnetic stirring for 16 hours. To the system obtained are added 20 ml of Milli-Q water, neutralized with hydrochloric acid and dialysed against distilled water. The product is recovered by lyophilization. The product swells in aqueous solutions.

### EXAMPLE 12

Preparation of Reticulated Polysaccharide

90 mg of tetrabutylammonium hyaluronan (the sodium salt thereof has MW:100000) are dissolved in 5 ml of dimethylsulphoxide in a 50 ml, round-bottomed flask with magnetic stirring at room temperature. Upon complete solubilisation 33 mg of dimethylaminopyridine and a catalytic quantity of tetrabutylammonium iodide are added. In another 10 ml, round-bottomed flask with magnetic stirring at room temperature, are dissolved 10 mg of C6-bromo hyaluronan, prepared as in example 3, in acid form, in 2 ml of dimethylsulphoxide. To the solution are added 4 mg of dimethylaminopyridine and all was added to the tetrabutylammonium hyaluronan. This is left to react at room temperature with gentle magnetic stirring for 16 hours. To the system obtained are added 20 ml of Milli-Q water, then neutralized with hydrochloric acid and dialysed against distilled water. The product is recovered by lyophilization. The product obtained is dispersed in aqueous solution and swells.

The esterified groups present in the reticulated polysaccharides obtained in examples 8-12 where determined with the saponification method described in “Quantitative Organic Analysis via Functional Groups”, John Wiley and Sons Publication, 4^{th} ed. The obtained results are reported hereunder.

| | Esterified groups |
|---|---|
| | (g sodium carboxylate/100 g |
| Example | polysaccharide) |
| Ex. 8 | 7 |
| Ex. 9 | 8 |
| Ex. 10 | 7 |
| Ex. 11 | 8 |
| Ex. 12 | 6 |

Further reticulated polysaccharides were prepared as follows.

### EXAMPLE 13

Preparation of Reticulated Polysaccharide

40 mg of tetrabutylammonium (21%)-sodium (79%) hyaluronan (the sodium salt of which has MW:1200000) are dissolved in 2 ml of dimethylsulphoxide in a round-bottomed flask with magnetic stirring at room temperature. Upon complete solubilisation, 33 mg of dimethylaminopyridine and a catalytic quantity of tetrabutylammonium iodide are added. In another 10 ml, round-bottomed flask with magnetic stirring at a temperature of 50° C., 30 mg of C6-bromo hyaluronan, prepared as in example 6, are dissolved in 2 ml of dimethylsulphoxide. The system is cooled to room temperature and added to the tetrabutylammonium-sodium hyaluronan solution. This is left to react at room temperature with gentle agitation for 16 hours. To the system obtained are added 20 ml of Milli-Q water, then neutralized with hydrochloric acid and dialysed against distilled water. The product swells in aqueous solutions and forms a high viscosity system.

### EXAMPLE 14

Preparation of Reticulated Polysaccharide

50 mg of tetrabutylammonium hyaluronan (the sodium salt thereof has MW:100000) are dissolved in 2 ml of dimethylsulphoxide in a three-necked flask under a current of nitrogen and with magnetic stirring at room temperature. Upon complete solubilisation, 12 mg of dimethylaminopyridine and a catalytic quantity of tetrabutylammonium iodide are added. In another 10 ml, round-bottomed flask with magnetic stirring at a temperature of 50° C., are dissolved 50 mg of C6-bromo laminaran prepared as in example 7, in 2 ml of dimethylsulphoxide. To the system are added 11 mg of dimethylaminopyridine and then all is added to the tetrabutylammonium hyaluronan solution. This is left to react at room temperature with gentle agitation for 2 hours and thirty minutes. Then another 11 mg of dimethylaminopyridine are added and left with agitation for a further hour. To the system obtained are added 20 ml of Milli-Q water, then neutralized with hydrochloric acid and dialysed against distilled water. The product is recovered by lyophilization. The product swells in aqueous solutions giving a very viscous system.

1. Regioselectively reticulated polysaccharide consisting of two polysaccharides, where the hydroxyl groups of carbon C-6 of the monosaccharide units of the first polysaccharide are regioselectively esterified with the carboxylic groups of the second polysaccharide and or with possible carboxylic groups of the first polysaccharide.

2. The polysaccharide according to claim 1 consisting of two polysaccharides, wherein the hydroxyl groups of carbon C-6 of the monosaccharide units of the first polysaccharide are totally or partially esterified.

3. The polysaccharide according to claim 2, wherein the number of hydroxyl groups esterified is comprised between 0.01% and 70%.

4. The polysaccharide according to any of claims 1-3, wherein the first polysaccharide is selected from the group consisting of hyaluronan, xanthan, carboxymethylcellulose with degrees of substitution less than 100%, carboxymethylchitin with degrees of substitution less than 100%, carboxymethylamylose with degrees of substitution less than 100%, carboxymethylguar with degrees of substitution less than 100%, scleroglucan, laminaran.

5. The polysaccharide according to any of claims 1-4, wherein the second polysaccharide is selected from the group consisting of hyaluronan, alginate, xanthan, carboxymethylcellulose, carboxymethylchitin, carboxymethylamylose, carboxymethylguar.

6. The polysaccharide according to any of claims 1-5, wherein the first polysaccharide and/or the second polysaccharide are further substituted.

7. The polysaccharide according to claim 6, wherein the first polysaccharide and/or the second polysaccharide are further substituted with drugs or biologically active substances.

8. The polysaccharide according to any of claims 1-7, wherein the carboxylic groups not involved in ester bonds are in acid or salt forms.

9. The polysaccharide according to any of claims 1-8, wherein the first polysaccharide is identical to the second polysaccharide.

10. The polysaccharide according to any of claims 1-9, wherein the first polysaccharide has a weight average molecular weight different from that of the second polysaccharide.

11. The polysaccharide according to claims 1-10, wherein both the first and the second polysaccharide are hyaluronan.

12. The polysaccharide according to claim 11, wherein the weight average molecular weight of the first polysaccharide is less than that of the second polysaccharide.

13. The polysaccharide according to any of claims 1-12 in the form of high viscosity solution.

14. The polysaccharide according to any of claims 1-12 in gel form.

15. The polysaccharide according to any of claims 1-12 in microsphere, thread, film forms.

16. Process for the preparation of reticulated polysaccharide described in any of claims 1-15, which comprises the following steps:
a) regioselective modification of the first polysaccharide through activation of the carbon C-6 of the monosaccharide units of said first polysaccharide;
b) formation of an ester bond between the carboxylic groups of the second polysaccharide and the C-6 atom of the first polysaccharide regioselectively activated obtained in a).

17. Process according to claim 16, wherein in step a) the activation of the carbon C-6 of the monosaccharide units of the first polysaccharide is obtained through halogenation of the carbon C-6, or through formation of C-6 O-alkylsulphonates, or C-6 O-arylsulphonates of said polysaccharide.

18. The process according to claim 17, wherein the halogenation is carried out by using a halogenating agent selected from the group consisting of: thionylbromide, thionylchloride, methanesulphonylchloride, methanesulphonylbromide, p-toluenesulphonylchloride, p-toluenesulphonylbromide, bis-trichloromethylcarbonate, phosgene, oxalyl bromide or chloride, optionally in mixtures.

19. The process according to any of claims 16-18, wherein in step b) the first regioselectively activated polysaccharide obtained in a) is suspended in an organic solvent and placed in contact with the second polysaccharide suspended in the same solvent, in the presence of a basic agent.

20. The process according to claim 19, wherein the organic solvent is selected from the group consisting of: N,N-dimethylformamide, dimethylsulphoxide, N-methylpyrrolidone.

21. The process according to claims 19-20, wherein the basic agent is chosen from either organic or inorganic bases.

22. Medicament comprising the reticulated polysaccharides described in any of claims 1-15.

23. Healthcare or surgical article comprising the reticulated polysaccharide described in any of claims 1-15.

24. Cosmetic article comprising the reticulated polysaccharide described in any of claims 1-15.

25. Use of the reticulated polysaccharide described in any of claims 1-15 in the pharmaceutical, cosmetic, healthcare, surgical fields.

26. Use of the reticulated polysaccharide described in any of claims 1-15 in the preparation of stationary phases for chromatography.

27. Use of the reticulated polysaccharide described in any of claims 1-15 in the preparation of plastic materials, composite materials, packing materials, adhesives, paints, industrial additives, rheologic modifiers.

28. Stationary chromatographic phases comprising the reticulated polysaccharide described in any of claims 1-15.
